(19) United States
(12) Patent Application Publication
(10) Pub. No.: US 2019/0374154 A1
(43) Pub. Date: Dec. 12, 2019

(54) Method, command, device and program to determine at least one brain network involved in carrying out a given process

(71) Applicants: Universite De Rennes 1, Rennes Cedex (FR); INSTITUT NATIONAL DE LA SANTE ET DE LA RECHERCHE MEDICALE, Paris (FR)

(72) Inventors: Fabrice Wendling, Rennes (FR); Mahmoud Hassan, Rennes (FR)

(21) Appl. No.: 16/488,489

(22) PCT Filed: Feb. 14, 2018
(86) PCT No.: PCT/EP2018/053726

§ 371 Date: Aug. 23, 2019

(30) Foreign Application Priority Data
Feb. 27, 2017 (FR) 1751585
Jul. 6, 2017 (FR) 1756378

(51) Int. Cl. A61B 5/00; G16H 50/20; A61B 5/04; A61B 5/0476; A61B 5/0484

(52) U.S. Cl. CPC A61B 5/4082; G16H 50/20; A61B 5/0484; A61B 5/0476; A61B 5/04012

(57) ABSTRACT

A method for determining a piece of data representing a cerebral marker. The piece of data is obtained from at least one brain network involved in performance of a given task. The is implemented by an electronic device and includes: obtaining data on encephalographic activities; processing the data on encephalographic activities, delivering at least one functional connectivity matrix representing connectivity between cortical sources derived from the data on encephalographic activities, each coefficient of the matrix representing connectivity between two cortical sources; statistical analysis of the at least one functional connectivity matrix delivering a probabilistic matrix of presence of at least one brain network; characterizing the at least one brain network on the basis of the at least one functional connectivity matrix and of the statistical analysis, delivering at least one brain network matrix; and obtaining a cerebral marker as a function of the at least one brain network matrix.

### 1. FIELD

The invention relates to a method, as well as to a device, for determining the involvement of brain networks in the implementation of processes. More particularly, the intervention relates to a device and a method for determining a correlation between the implementation of a process (or a task) and the activation and/or the connection of brain networks. Yet more specifically, the invention quantifies the level of interaction between brain networks (functional connectivity) during the performance of a given task.

### 2. PRIOR ART

It is believed that cognitive deficiency in Parkinson's Disease is related to impaired functional brain connectivity. To date, the changes in cognitive functions in Parkinson's Disease have never been explored with dense EEG in order to establish a relationship between the degree of cognitive deficiency on the one hand and deterioration in the functional connectivity of brain networks on the other hand.

### 3. SUMMARY OF THE INVENTION

The proposed technique does not raise these problems of the prior art. More particularly, it brings a simple solution to the problems identified here above. More particularly, the invention relates to a method for determining a piece of data representing a cerebral marker, said piece of data being obtained from at least one brain network involved in the performance of a given task, the method being implemented by means of an electronic device comprising means to obtain data on encephalographic activity. According to the invention, this method comprises the succession of the following steps:
a step of processing data on encephalographic activities, delivering at least one functional connectivity matrix representing connectivity between cortical sources derived from said data on encephalographic activities, each coefficient of said matrix representing connectivity between two cortical sources;
a step of statistical analysis of said at least one functional connectivity matrix delivering a probabilistic matrix of presence of at least one brain network;
a step of characterizing said at least one brain network on the basis of said at least functional connectivity matrix and of said statistical analysis, delivering at least one brain network matrix;
a step of obtaining a cerebral marker as a function of said at least one brain network matrix.

According to at least one particular embodiment, said step of obtaining a cerebral marker (EWCI) as a function of said at least one brain network matrix comprises the application of the following formula:

$$EWCI = ( {\sum }_{i}^{N} {W}_{i} ) \times 100$$

Wherein:
N represents the number of edges of the brain network;
Wᵢ represents the weight of the edge i in the brain network.

According to one particular embodiment, said step of processing data on encephalographic activities comprises:
a step of pre-processing signals coming from a surface electronic device for measuring encephalographic signals as a function of at least one pre-processing parameter;
a step of determining a plurality of cortical sources producing said encephalographic signals;
a plurality of steps for the analysis of pairwise connectivities that comprises, for each pair of cortical sources, at least one step of determining a connectivity between the two sources of said pair;
said step of processing data on encephalographic activities delivering a square matrix, called a functional connectivity matrix, comprising, for each cortical source, a value of connectivity with all the other pre-determined cortical sources.

According to one particular characteristic, said step of statistical analysis of said at least one functional connectivity matrix comprises, for a current functional connectivity matrix, the implementing of a method of network-based statistical analysis called the NBS method.

According to one particular characteristic, said step of statistical analysis of said at least one functional connectivity matrix comprises, for a current functional connectivity matrix:
a step of analysis of covariance of each coefficient of the current functional connectivity matrix, delivering a probabilistic matrix, wherein each coefficient is represented by a probability p of rejecting the null hypothesis for an edge of the brain network associated with said coefficient of the current functional connectivity matrix;
a step of application of a component-forming threshold T on each coefficient p of said probabilistic matrix delivering a thresholded matrix;
a step of obtaining a size of components, representing the number of edges of said brain network, on the basis of said thresholded matrix;
a step of the obtaining, by means of permutation tests, of the maximum size of the randomly defined components;
a step of acceptance when the maximum size of randomly defined components differs from the size of preliminarily obtained components by a predefined acceptance threshold.

According to one particular characteristic, the component-forming threshold T ranges from 0.01 to 0.001.

According to one particular embodiment, the component-forming threshold T is equal to 0.005.

According to another aspect, the invention also relates to an electronic device for determining a piece of data representing a cerebral marker, said piece of data being obtained from at least one brain network involved in carrying out a given task, the device comprising means for obtaining data on encephalographic activities. According to the invention, such a device comprises:
means for processing data on encephalographic activities, delivering at least one functional connectivity matrix, representing connectivity between cortical sources derived from said data on encephalographic activities, each coefficient of said matrix representing a connectivity between two cortical sources;
means of statistical analysis of said at least one functional connectivity matrix delivering a probabilistic matrix of presence of at least one brain network;
means for characterizing said at least one network obtained from said at least one functional connectivity matrix and from said statistical analysis delivering at least one brain network matrix;
means for obtaining a statistical marker as a function of said at least one brain network matrix.

According to a preferred application, the different steps of the methods according to the invention are implemented by one or more computer software programs comprising software instructions to be executed by a data processor of a relay module according to the invention and designed to command the execution of the different steps of the methods.

The invention is therefore also aimed at providing a program capable of being executed by a computer or by a data processor, this program comprising instructions to command the execution of the steps of a method as mentioned here above.

This program can use any programming language whatsoever and can be in the form of source code, object code or intermediate code between source code and object code such as in a partially compiled form or in any other desirable form whatsoever.

The invention is also aimed at providing an information carrier or medium readable by a data processor, and comprising instructions of a program as mentioned here above.

The information medium can be any entity or device whatsoever capable of storing the program. For example, the medium can comprise a storage means such as a ROM, for example, a CD ROM or microelectronic circuit ROM or again a magnetic recording means, for example a floppy disk or a hard disk drive.

Besides, the information medium can be a transmissible medium such as an electrical or optical signal, that can be conveyed by an electrical or optical cable, by radio or by other means. The program according to the invention can be especially downloaded from an Internet type network.

As an alternative, the information medium can be an integrated circuit into which the program is incorporated, the circuit being adapted to executing or to being used in the execution of the method in question.

According to one embodiment, the proposed technique is implemented by means of software and/or hardware components. In this respect, the term “module” can correspond in this document equally well to a software component and to a hardware component or to a set of hardware and software components.

A software component corresponds to one or more computer programs, one or more sub-programs of a program or more generally to any element of a program or a piece of software capable of implementing a function or a set of functions according to what is described here below for the module concerned. Such a software component is executed by a data processor of a physical entity (terminal, server, gateway, router etc) and is capable of accessing the hardware resources of this physical entity (memories, recording media, communications buses, input/output electronic boards, user interfaces etc).

In the same way, a hardware component corresponds to any element of a hardware assembly capable of implementing a function or a set of functions according to what is described here below for the module concerned. It can be a programmable hardware component or a component with an integrated processor for the execution of software, for example, an integrated circuit, smart card, a memory card, an electronic board for the execution of firmware etc.

Each component of the system described here of course implements its own software modules.

The different embodiments mentioned here above can be combined with one another to implement the proposed technique.

### 4. DRAWINGS

Other features and advantages of the invention shall appear more clearly from the following description of a preferred embodiment, given by way of a simple illustratory and non-exhaustive example and from the appended drawings, of which:

FIG. 1 presents a comprehensive view of the application of the method in which the invention is situated;

FIG. 2 presents the results of frequency-based and network-based analyses;

FIG. 3 illustrates the functional connection sub-networks showing a significant difference between the three groups at alpha 2 with T=0.01;

FIG. 4 illustrates the analysis of the network edges and shows a significant difference between the three groups at alpha 1. The functional connection sub-networks show a significant difference between the three groups at alpha 2 with T=0.001;

FIG. 5 is a graph of association between the cognitive score and the connectivity index for A) G1, G2 and G3 and B) G1 and G2;

FIG. 6 describes a device for implementing the proposed techniques;

FIG. 7 is a general illustration of the method of the invention.

### 5. DESCRIPTION

### 5.1. Reminders of the Principle

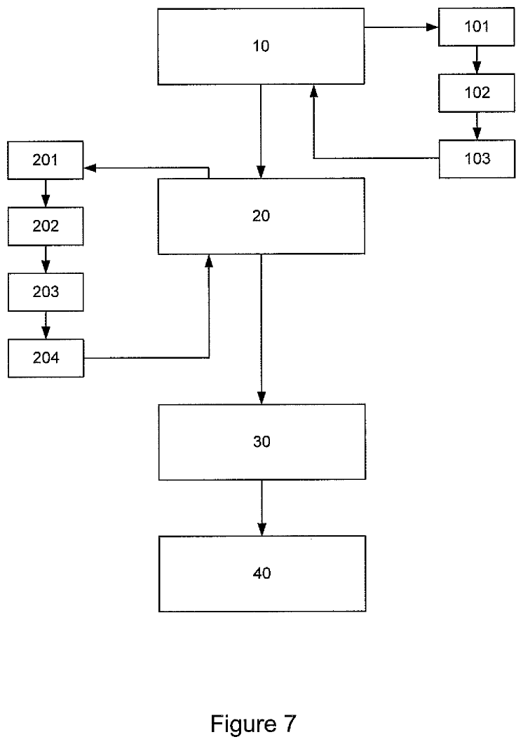

The invention relates to a method and a device to identify impaired brain networks associated with cognitive phenotypes in Parkinson's Disease (and other diseases) using dense EEG data recorded at rest, with eyes closed. The invention is aimed at constructing at least one static marker that will probably be used by another method or device to identify the presence or absence of early signs of appearance of the disease. The inventors have looked for a solution making it possible to obtain a synthetic view, in a given index, of the degree of functional connectivity of brain networks implemented during the performance of a given task which, in the context of the present invention, may be a task requiring action on the part of the individual, or else a task where one remains still without performing any action, i.e. an action where one is in a state of rest. To construct this representative index (connectivity index, cerebral marker), the inventors have applied a certain number of computation phases and processing steps that are described here below. In general, with reference to FIG. 7, the invention relates to a method for determining a piece of data representing a cerebral marker, the piece of data being obtained from at least one brain network involved in the performance of a given task, the method comprising:
a step of processing (10) data on encephalographic activities, delivering at least one functional connectivity matrix representing connectivity between cortical sources, derived from said data on encephalographic activities, each coefficient of the matrix representing a connectivity between two cortical sources;
a step of statistical analysis (20) of functional connectivity matrices delivering a probabilistic matrix of presence of at least one brain network;
a step of characterization (30) of brain networks on the basis of matrices of functional connectivity and of statistical analysis (20), delivering at least one brain network matrix.

In the implementing of this technique, the step of processing encephalographic data described here below comprises:
a step of pre-processing (101) signals coming from a surface electronic device for measuring encephalographic signals as a function of at least one pre-processing parameter; such a device is for example a high-density encephalographic device;
a step of determining (102) a plurality of cortical sources producing said encephalographic signals; this is the implementing of an algorithm for reconstructing cortical sources to determine the origin of the recorded signal;
a plurality of steps for analyzing (103) pairwise connectivity that comprises, for each pair of cortical sources, at least one step of determining connectivity between the two sources of the pair.

The step of processing data on encephalographic activities delivers a square matrix called a functional connectivity matrix comprising, for each cortical source, a value of connectivity with all the other predetermined cortical sources.

The step of statistical analysis (20) implemented on the basis of matrices of functional connectivity comprises, for its part, for a current functional connectivity matrix:
a step of analysis of covarance (ANCOVA) (201) of each coefficient of the current functional connectivity matrix, delivering a probabilistic matrix, wherein each coefficient represents a probability p of rejecting the null hypothesis for a brain network edge associated with said coefficient of the current functional connectivity matrix;
a step of application (202) of a component-forming threshold T on each coefficient p of said probabilistic matrix, delivering a thresholded matrix;
a step of obtaining (203) a size of components representing the number of edges of said brain network on the basis of said threshold matrix;
a step of obtaining (204) the maximum size of the randomly defined components by means of permutation tests;
a step of acceptance, when the maximum size of the randomly defined components differs from the size of preliminarily obtained components by a pre-defined acceptance threshold.

This statistical analysis eliminates data that might be not representative of the presence of a brain network. These different steps make it possible ultimately to characterize the brain networks that come from the execution of the task (in this case a task of resting) and then, by means of the characterized networks, to compute the cerebral marker associated with these networks (the connectivity index).

### 5.2. Description of a Case of Application

Pathological disturbances of the brain are rarely limited to a single region. The local dysfunction often propagates via axonal paths and affects other regions, leading to large-scale network impairment. In recent years, the identification of impairment of functional and structural networks through neuro-imaging data has become one of the most promising prospects in brain disease research. Indeed, neuro-imaging helps in the investigation of pathophysiological mechanisms in vivo, and the results derived from previous studies show that brain network topology tends to shape neural responses to damage. In graph-theory approaches, brain networks are characterized as sets of nodes (brain regions) connected by edges. Once the nodes and the edges are defined on the basis of neuro-imaging data, the network topological properties (organization) can be studied by graph-theory metrics and the functional connectivity can be studied by network-based statistics. By using different neuro-imaging techniques (functional magnetic resonance imaging (fMRI) magneto/electro-encephalography (MEG/EEG), these combined approaches are used to characterize functional changes associated with states such as Alzheimer's disease, Parkinson's disease, Huntingdon's disease, epilepsy, schizophrenia, autism and the like.

Parkinson's disease is the second most widespread neuro-degenerative disease after Alzheimer's and affects more than 1% of individuals aged more than 60 years. In addition to the hallmark motor symptoms, cognitive deficiency or deficiency is common in Parkinson's disease. These symptoms are however heterogeneous in their clinical presentation and their progress. The early detection and quantitative assessment of these cognitive deficiencys are a crucial clinical problem not only for characterizing the disease but also for studying its progress. Several studies have already reported the impairment of brain network organization and functional connectivity associated with cognitive deficiency in Parkinson's disease by using FMRI, MEG and standard EEG. Until now, the changes related to cognitive functions of brain connectivity in Parkinson's disease have never been explored with dense EEG in order to establish a relationship between i) the degree of cognitive deficiency on the one hand and ii) spatially localized impairment of functional connectivity of brain networks on the other hand.

The inventors have recorded a dense EEG in a resting state, with eyes closed, in Parkinson's disease patients, whose cognitive profile has been identified by a cluster analysis of the results of an extensive battery of neuro-psychological tests. The main goal of the inventors is to detect impairments in these functional networks according to the severity of the cognitive deficiency. To this end, functional connectivity is examined by using an “EEG source connectivity” method. As compared with fMRI studies of functional connectivity, a unique advantage of this method is that the networks can be directly identified at the cerebral cortex level from scalp EEG recordings, which consist of the direct measurement of neural activity, in contrast to blood oxygen level dependent (BOLD) signals. The inventors' main hypothesis is that EEG connectivity gradually deteriorates as the cognitive deficiency worsens. More specifically, the inventors have assumed that the parameters of brain network organization differ according to the the cognitive state of the individuals and that functional connectivity is impaired to a greater extent among individuals with cognitive deficiency then among individuals who are cognitively intact or have lesser cognitive deficiency. From this assumption, the inventors have sought to construct an index (a clue) that can be used to quantify this functional connectivity. Thus, the value of the methods proposed and described lies firstly in the capacity to identify characteristic networks in populations of individuals and, secondly, from these networks, to compute an index, the index being a result to characterize the functional connectivity of the networks. The proposed methods use the determining of functional networks using recorded data on an individual and using methods for the analysis of similarities and differences in these networks. The connectivity index that is computed on these networks gives a characteristic value from the weight of a large number of connections on the pairs of the networks: the index of connectivity is therefore considered to be the cerebral marker, of statistical origin, related to the application of the given task for an individual. Detailed explanations are given here below for specific embodiments.

According to one example of implementation of the proposed technique, described here below, three groups of individuals suffering from Parkinson's disease (N=124), with different cognitive phenotypes obtained from a data-driven cluster analysis, are studied: G1) cognitively intact individuals (N=63), G2) individuals with mild cognitive deficiency (N=46), and G3) individuals with severe cognitive deficiency (N=15). Functional brain networks are identified using a method for determining dense EEG source connectivity. A pairwise functional connectivity is computed for 68 brain regions in different EEG frequency bands. Statistics on brain networks are obtained both at a comprehensive level (network topology) and at a local level (inter-regional connections). The connectivity index (cerebral marker) is then computed on the basis of a certain number of pre-determined connectivity networks.

### 5.3. Methods

### 5.3.1. Data Acquisition and Pre-Processing

This is the first sub-step of the step of processing data on encephalographic activities. According to the invention, dense EEGs are recorded with a cap provided with 128 channels including 122 scalp electrodes distributed according to the 10-05 international system, two electrocardiogram electrodes and four bilateral electro-oculogram electrodes (EOG) for vertical and horizontal movements. The impedance of the electrodes is kept at 10 kΩ. The data, in this embodiment, are collected in a state of rest, with eyes closed, for 10 minutes using the BrainVision Recorder (Brain Products®) software. According to this example of an embodiment, the subjects were asked to do nothing and relax. The signals were sampled at 512 Hz and bandpass-filtered between 1 Hz and 45 Hz. For each participant, the inventors selected the maximum number of artefact-free, four-second segments for the analyses. An atlas-based approach is used to project EEG sensor signals onto an anatomical frame consisting of 68 cortical regions identified by means of the Desikan-Killiany atlas (Desikan et al., 2006) using the Freesurfer software (http://freesurfer.net/). To this end, an MRI model and EEG data are recorded with identification of the same anatomical references (pre-auricular left and right points and nasion). A realistic head model was constructed by segmenting the MRI image using Freesurfer. The lead field matrix was then computed for a cortical mesh with 15,000 vertices by means of Brainstorm and OpenMEEG.

### 5.3.2. Power Spectrum Analysis

This is the second sub-step of the step of processing data on encephalographic activities. In this step, the method comprises the use of a standard Fast Fourier transform (FFT for power spectrum analysis with the Welch technique and Hanning windowing function (two-second epoch and 50% overlap). A relative power spectrum was computed for each frequency band [delta (0.5-4 Hz); theta (4-8 Hz); alpha 1 (8-10 Hz); alpha 2 (10-13 Hz); beta (13-30 Hz); gamma (30-45 Hz)], with a frequency resolution of 0.5 Hz.

### 5.3.3. Analysis of Functional Connectivity

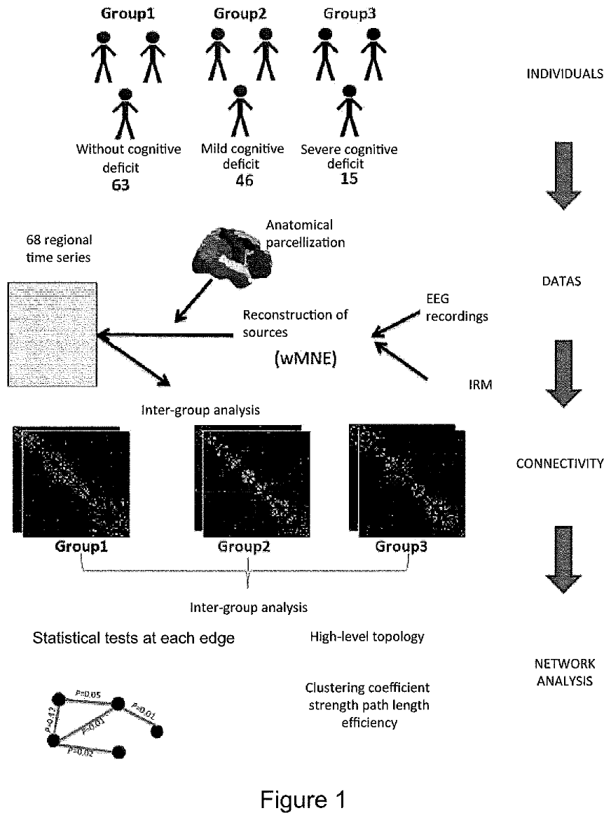

This is the third sub-step of the step of processing data on encephalographic activities. In this step, functional connectivity matrices are constructed using a “EEG source connectivity” that comprises two main steps: i) resolving the EEG inverse problem to reconstruct the temporal dynamics of the cortical regions and ii) measuring the functional connectivity between these reconstructed regional time series (FIG. 1). The weighted Minimum Norm Estimate (wMNE) is used to reconstruct the dynamics of the cortical sources. We then compute the functional connectivity between the reconstructed sources by using the phase synchronization (PS) method. In order to measure the PS, the phase locking value (PLV) method is used as described. This value (range between 0 and 1) reflects the precise interactions between two oscillatory signals through quantification of the phase relationships. The PLVs are estimated at six frequency bands [delta (0.5-4 Hz); theta (4-8 Hz); alpha 1 (8-10 Hz); alpha 2 (10-13 Hz); beta (13-30 Hz); gamma (30-45 Hz)]. The choice of wMNE/PLV is supported by two comparison analyses performed. These analyses have indicated the superiority of wMNE/PLV over other combinations of inversion/connectivity in precisely identifying the cortical brain networks from scalp EEG during cognitive activity or epileptic activity. The inversion solutions are computed using Brainstorm. The network measurements and network visualization are done using BCT and EEGNET respectively.

### 5.3.4. Network Analysis

This step is used to prepare the obtaining of connectivity networks, especially by statistical analysis. Networks can be illustrated by graphs which are sets of nodes (brain regions) and edges (connectivity values) between these nodes. The method comprises the construction of 68-node graphs (i.e. the 68 cortical regions identified here above) and uses all the information from the functional connectivity matrix (phase threshold value). This gives fully connected, weighted and undirected networks in which the connection strength between each pair of vertices (i.e the weights) is defined as their connectivity value.

Several metrics can be computed to characterize weighted networks. Here, it is proposed to examine a network analysis at two levels: i) the comprehensive or global level reflects the overall network organization where several measurement are computed including the path length (P_{L}), (the clustering coefficient C_{C}), the strength (Str) and the overall efficiency (E_{G}) (greater detail is provided in the illustratory embodiment) and ii) the edgewise level reflects the functional connectivity through the measurement of each of the correlation values (weights) between the different brain regions. All the network measurements referred to here above depend on the weights of the edges. They are therefore standardized. They are expressed as a function of measurements computed from random networks. Five hundred random substitution networks derived from the original networks are generated by the random reshuffling of the weights of the edges. The standardized values are computed by dividing the original value by the average of the values computed on the random graphs.

### 5.3.5. Statistical Analyses

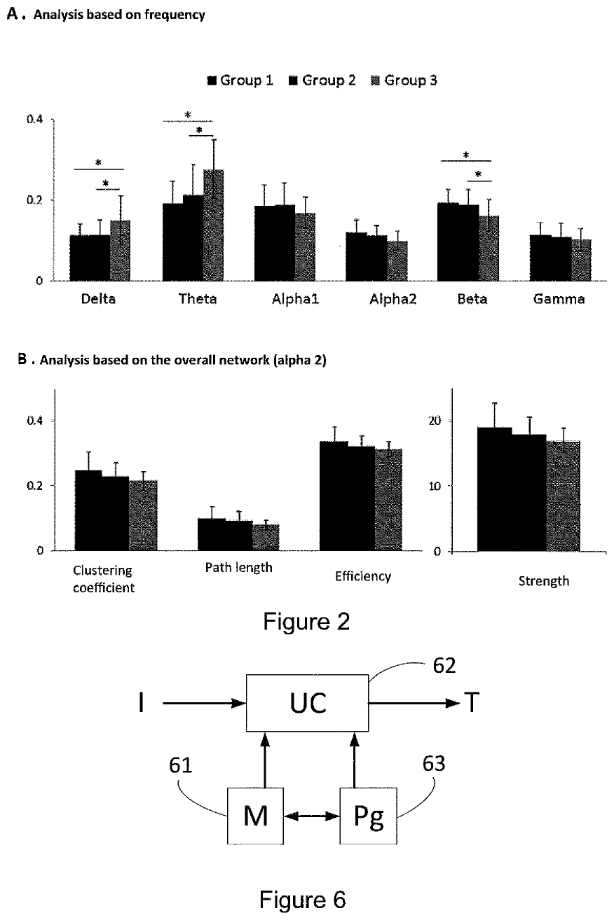

The edgewise connectivity is characterized by using network-based statistics. To compute the network-based statistics, an ANCOVA analysis is adapted to each of the (68²−68)/2=2278 edges (phase synchronization values) in the (68×68) functional connectivity matrix giving a p value matrix indicating the probability of rejecting the null hypothesis for each edge. A threshold matrix is generated by applying, to each value p, a component-forming threshold, T, and the size of each connected element in this thresholded matrix is obtained. This size of the components is then compared with the size obtained for a null distribution of maximum component sizes obtained by using a permutation test in order to obtain values p corrected for multiple comparisons. The NBS method finds sub-networks of connections considerably greater than might be expected. In compliance with this result, the inventors have reported results for a threshold that retains only the edges with p<0.005. The results at higher threshold values (p<0.01) and lower threshold values (p<0.001) are reported in FIG. 2 and respectively in the illustratory embodiment to show sensitivity to sets of parameters.

The age and duration of formal education are entered as confounding factors in ANCOVA for spectral analyses and connectivity analyses. The statistical analyses are performed by using the SPSS Statistics 20.0 (IBM Corporation) software package. A significance level of 0.01 (two-tailed) is applied. Corrections for multiple tests are applied using the Bonferroni approach.

### 5.4. Characteristics of Networks Obtained

### 5.4.1. Power-Based Analysis

The results of the frequency-based analysis are recapitulated in FIG. 2a. In the frequency bands alpha 1, alpha 2, beta and gamma, there is a progressive decrease in the power spectral density as the cognitive deficiency worsens (from G1 to G3). Conversely, in the frequency bands delta and theta, there is an increase in the power spectral density as the cognitive deficiency worsens (from G1 to G3). Significant differences are observed between G1 and G3 and between G2 and G3 in the delta, theta and beta frequency bands (p<0.01 Bonferroni corrected for each comparison). No significant difference is observed between G1 and G2 whatever the frequency band.

### 5.4.2. Network-Based Topology Analysis

The four metrics reflecting the overall topology of the networks (P_{L}, C_{C}, Str and E_{G}) are computed on the weighted undirected graphs obtained for each subject of each group in all the frequency bands. The results tend to decrease as the cognitive deficiency worsens (from G1 to G3), in all the frequency bands, without any significant difference. A typical example of the results obtained in the alpha 2 frequency band is presented in FIG. 2. As compared with the other frequency bands, the results at alpha 2 demonstrate the lowest values p (non-significant values) (p=0.063, p=0.067, p=0.1 and p=0.08 for C_{C}, Str, P_{L} and E_{G} respectively, ANCOVA corrected by Bonferroni test).

### 5.4.3. Network Edgewise Analysis

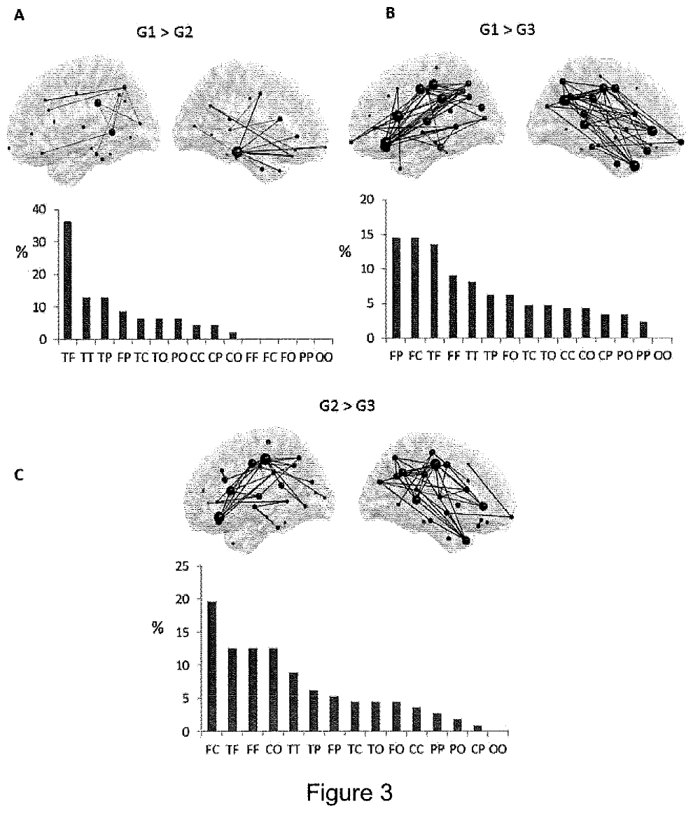

FIG. 3 shows the results of the edgewise analysis made by using the NBS toolbox. The statistical tests (ANCOVA corrected by permutation test) are applied to each connection in the networks computed at all the frequency bands (delta, theta, alpha 1, alpha 2, beta and gamma). Significant differences are found solely between the networks computed in the EEG alpha band (alpha 1 and alpha 2).

With regard to the alpha 2 networks, the difference between G1 and G2 of a connected component comprising 49 edges and 36 regions has proved to be statistically significant (p=0.03, corrected in using the permutation test, FIG. 3A). For all these edges, the connectivity is considerably lower in G2 than in G1. For a better understanding of the regional distribution of these connections, the inventors have classified each region as belonging to one of five large areas of the scalp: frontal, temporal, occipital, or central. The inventors have then classified each edge in the affected sub-network on the basis of the areas that it connects (for example frontal-temporal, temporal-parietal etc.) and counted the proportion of edges falling into each category. When G1 and G2 are compared, the connections most reduced in G2 are the frontal-temporal connections (FIG. 3A, «TF», 36%). Similar results are obtained on different threshold values (see FIG. 2 and FIG. 3 for this illustratory embodiment).

When G2 and G3 are compared, a connected component comprising 125 edges and 57 regions appears in a statistically significant way (p<0.001, corrected by using the permutation test, FIG. 2). For all the edges, the functional connectivity is considerably reduced in G3. Most of these impaired connections were the frontal-central (20%), temporal-frontal (12%), frontal-frontal (12%) and occipital-central (12%) connections. Similar results are obtained from different threshold values (see FIG. 2 and FIG. 3, for this illustratory embodiment).

A connected component comprising 229 edges and 57 regions emerges in a statistically significant way (p<0.001, corrected by the permutation test, FIG. 3C). Most of these decreased connections are the parietal-frontal (14%), frontal-central (14%) and temporal-frontal (13%) connections. Similar results are obtained on different threshold values (see FIG. 2 and FIG. 3, for this illustratory embodiment).

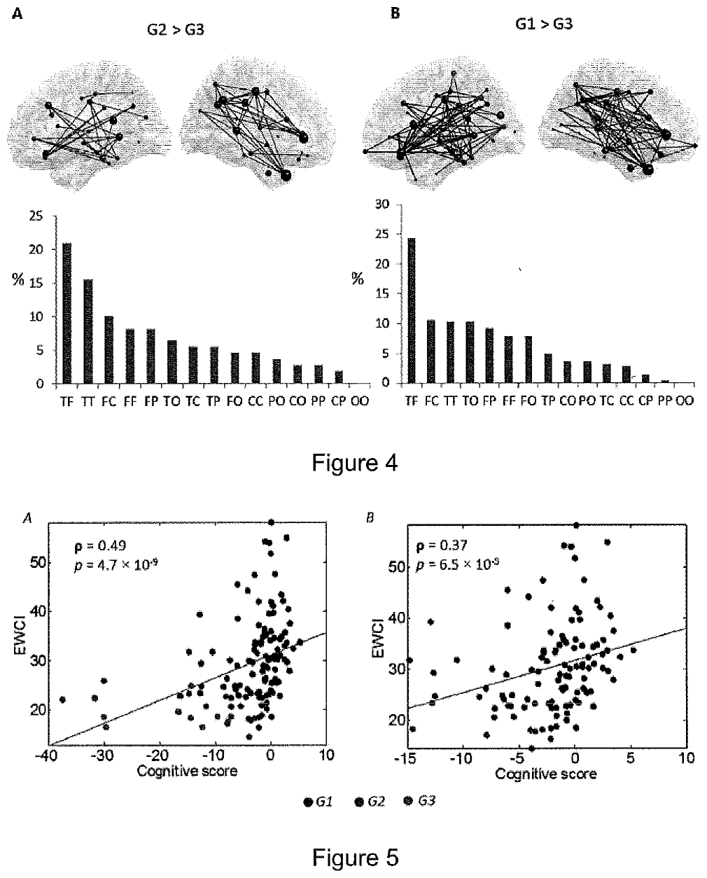

For the alpha 1 networks, the results show a statistically significant difference between G2 and G3 with a component of 60 nodes and 320 edges (p<0.001, FIG. 4A). These impairments relate chiefly to the temporal-frontal (20%), temporal-temporal (15%) and frontal-central (10%) connections.

In addition, a connected component comprising 123 edges and 47 regions shows significant differences between G1 and G3 (p=0.004, FIG. 4B). Most of these decreased connections are temporal-frontal (24%) and temporal-frontal (10%). No significant difference is observed between G1 and G2 in the alpha 1 frequency band.

### 5.4.4. Correlations Between Brain Connectivity and Performance During Neuro-Psychological Tests

To asses the relationships between functional connectivity and cognitive performance of individuals suffering from Parkinson's disease, the inventors have concentrated on the sub-network showing a significant difference between G1 and G2 (FIG. 3A). The inventors have concluded that these 49 edges are the most relevant for detecting a marker of cognitive deficiency. For each network, an edge connectivity index (EWCI) is computed as a sum of the weights of significant sub-networks:

$$EWCI = ( {\sum }_{i = 1}^{N} {W}_{i} ) \times 100$$

where Wᵢ represents the weight of the edge i in the significant sub-network and N is the number of edges in the sub-network (N=49 in this case). For the correlation analysis, the inventors have used the three most discriminant neuro-psychological tests identified by discriminant factor analysis. It includes the number of correct responses in the symbol digit modalities test (SDMT), the number of errors in the Stroop test and animal fluency in 60 s. Z scores are computed for each of these tests and the cognitive score used for the correlation analysis (Spearman's ρ) is the sum of these Z scores. The results are illustrated in FIG. 5. When all the groups are considered, the EWCI is significantly correlated with the cognitive score (p=0.49, p<0.01), FIG. 5A. To make sure that the correlation is not only driven by G3 (as can be seen in the figure), the inventors have computed the correlation between EWCI and the cognitive score for G1 and G2: the result show that the association remains significant (p=0.37, p<0.01), FIG. 5B.

### 5.5. Illustratory Embodiments and Results

FIG. 1: Structure of the investigation. The individuals are classified by their cognitive performance: 1) cognitively intact individuals, 2) individuals with mild cognitive deficiency and 3) individuals with severe cognitive deficiency. Data: Dense EEGs were recoded using 128 electrodes during the resting state (eyes closed). The MRIs of the subjects are also available. The cortical sources are reconstructed by resolving the inverse problem using the weighted Minimum Norm Estimate (wMNE) method. An anatomical parcellation is applied to the MRI template producing 68 regions of interest (the Desikan-Killany atlas) computed using Freesurfer and then imported into Brainstorm for another processing operation. The functional connectivity is computed between the 68 regional temporal series using the phase-locking value (PLV) method in six frequency bands: delta (0.5-4 Hz); theta (4-8 Hz); alpha 1 (8-10 Hz); alpha 2 (10-13 Hz); beta (13-30 Hz); gamma (30-45 Hz). The connectivity matrices are compared between the groups using two levels of network analysis i) high-level topology where the inventors have computed four network metrics: clustering coefficient, strength, characteristic path length and overall efficiency and ii) edgewise analysis where the inventors have carried out statistical analysis between the groups at each connection in the network using the network-based statistics (NBS) approach.

FIG. 2: A. frequency-based analysis: mean±standard deviation values of the power spectral density for each group of individuals in six frequency bands: delta (0.5-4 Hz); theta (4-8 Hz); alpha 1 (8-10 Hz); alpha 2 (10-13 Hz); beta (13-30 Hz); gamma (30-45 Hz). B. Analysis of overall topology: mean±standard deviation values of four computed network measurements: cluster coefficient, strength, path length and overall efficiency. This typical example corresponds to the metrics computed on the weighted undirected graphs obtained for each subject of each group in the alpha 2 frequency band. The * designates a value of p<0.01, Bonferroni corrected.

FIG. 3: Edgewise analysis (alpha 2). Sub-networks of functional connections showing significant differences between the three groups at alpha 2. At each part, the top row presents graph-based representations of these sub-networks, each region being represented by a red sphere plotted according to the stereotactic coordinates of its centroid, and each supra-threshold edge is represented by a dark green line. The size of the node represents the number of significantly different connections from the node itself. For all the edges, the connectivity is higher in G1>G2 (A), G1>G3 (B) and G2>G3 (C). The bottom row presents the proportion (%) of each type of connection in each sub-network as categorized according to the lobes that each edge interconnects. F: frontal, T: temporal, P: parietal, C: central and O: occipital.

FIG. 4: Edgewise (alpha 1). Sub-networks of functional connections showing a significant difference between the three groups at alpha 1. In each part, the top row presents graph-based representations of these sub-networks, each region being represented by a red sphere plotted according to the stereotactic coordinates of its centroid, and each supra-threshold edge being represented by a dark green line. The size of the node represents the number of significantly different connections from the node itself. For all the edges, the connectivity was the highest in G2>G3 (A) and G1>G3 (B). The bottom row presents the proportion (%) of each type of connection in each sub-network as categorized according to the lobes that each edge interconnects. F: frontal, T: temporal, P: parietal, C: central and O: occipital.

FIG. 5: Diagram of dispersion of the association between the cognitive score and the connectivity index of the edges for A) G1, G2 and G3 and B) G1 and G2.

### 5.6. Devices for the Estimation of Networks and the Obtaining of Statistical Markers

The description also proposes a device to estimate networks and obtain statistical markers. The device can be specifically designed to estimate networks and obtain statistical markers, or it can be any electronic device comprising a non-transient computer-readable medium and at least one processor configured by computer-readable instructions stored in the computer-readable medium to implement any unspecified method of the description.

According to one embodiment illustrated in FIG. 6, the device for estimating the camera pose comprises a central processing unit (CPU) 62, a random-access memory (RAM) 61, a read-only memory (ROM) 63, a storage device that is connected by means of a bus in such a way that they can carry out communications with one another.

The CPU commands the totality of the device in executing a program loaded into the RAM. The CPU also carries out various functions in executing one program or one of of the programs (an application or one of the applications) loaded into the RAM.

The RAM stores various sorts of data and/or programs.

The ROM also stores various sorts of data and/or programs (Pg).

The storage device, for example a hard disk drive reader, an SD card, a USB memory and so on and so forth, also stores various types of data and/or a program or programs.

The device carries out a method for estimating networks and obtaining statistical markers as a consequence of the the execution, by the CPU, of instructions written to programs loaded into the RAM, the programs being read from the ROM and the storage device and loaded into the RAM.

More specifically, the device can be a server, a computer, a tablet, a smartphone or a medical device in this smartphone. The device comprises at least one input adapted to receiving data coming from a dense EEG, at least one other input parameter, the processor or processors for estimating networks and obtaining statistical markers and at least one output adapted to outputting the data associated with the markers or the networks.

The invention also relates to a computer program product comprising a program code recorded on a computer-readable non-transient storage medium, the computer-executable program code, when it is executed, performing the method to estimate a camera pose. The computer program product can be recorded on a CD, a hard disk drive, a flash memory or any other appropriate computer-readable medium. It can also be downloaded from the Internet and installed in a device so as to estimate a camera pose as explained here above.

1. A method for determining a piece of data representing a cerebral marker, said piece of data being obtained from at least one brain network involved in performance of a given task, the method being implemented by an electronic device comprising elements to obtain data on encephalographic activities, the method comprising:
processing the obtained data on encephalographic activities, delivering at least one functional connectivity matrix representing connectivity between cortical sources derived from said data on encephalographic activities, each coefficient of said matrix representing connectivity between two cortical sources;
statistical analysis of said at least one functional connectivity matrix delivering a probabilistic matrix of presence of at least one brain network;
characterizing said at least one brain network on the basis of said at least one functional connectivity matrix and of said statistical analysis, delivering at least one brain network matrix; and
obtaining a cerebral marker as a function of said at least one brain network matrix.

2. The method according to claim 1, wherein said obtaining a cerebral marker (EWCI) as a function of said at least one brain network matrix comprises application of the following formula:
$$EWCI = ( {\sum }_{i}^{N} {W}_{i} ) \times 100$$
wherein:
N represents a number of edges of the brain network;
Wᵢ represents weight of an edge i in a matrix of a brain network.

3. The method according to claim 1, wherein said processing data on encephalographic activities comprises:
pre-processing signals coming from a surface electronic device, which measures encephalographic signals, as a function of at least one pre-processing parameter;
determining a plurality of cortical sources producing said encephalographic signals;
a plurality of acts of analyzing pairwise connectivities that comprises, for each pair of cortical sources, at least one act of determining a connectivity between the two sources of said pair;
said act of processing data on encephalographic activities delivering a square matrix, called a functional connectivity matrix, comprising, for each cortical source, a value of connectivity with all the other pre-determined cortical sources.

4. The method according to claim 1, wherein said statistical analysis of said at least one functional connectivity matrix comprises, for a current functional connectivity matrix, implementing a method of network-based statistical analysis called an NBS method.

5. The method according to claim 1, wherein said statistical analysis of said at least one functional connectivity matrix comprises, for a current functional connectivity matrix:
analysis of covariance of each coefficient of the current functional connectivity matrix, delivering a probabilistic matrix, wherein each coefficient is represented by a probability p of rejecting a null hypothesis for an edge of a brain network associated with said coefficient of the current functional connectivity matrix;
application of a component-forming threshold T on each coefficient p of said probabilistic matrix, delivering a thresholded matrix;
obtaining a size of components, representing the number of edges of said brain network, on the basis of said thresholded matrix;
obtaining, by permutation tests, of a maximum size of randomly defined components;
acceptance when the maximum size of randomly defined components differs from the size of preliminarily obtained components by a predefined acceptance threshold.

6. The method according to claim 5, wherein the component-forming threshold T ranges from 0.01 to 0.001.

7. The method according to claim 5, wherein the component-forming threshold T is equal to 0.005.

8. An electronic device for determining a piece of data representing a cerebral marker, said piece of data being obtained from at least one brain network involved in carrying out a given task, the device comprising:
a processor; and
a non-transitory computer-readable medium comprising instructions stored thereon, which when executed by the processor configure the electronic device to perform acts comprising:
obtaining data on encephalographic activities;
processing the data on encephalographic activities, delivering at least one functional connectivity matrix, representing connectivity between cortical sources derived from said data on encephalographic activities, each coefficient of said matrix representing a connectivity between two cortical sources;
statistical analysis of said at least one functional connectivity matrix delivering a probabilistic matrix of presence of at least one brain network;
characterizing said at least one network obtained from said at least one functional connectivity matrix and from said statistical analysis delivering at least one brain network matrix; and
obtaining a statistical marker as a function of said at least one brain network matrix.

9. A non-transitory computer-readable medium comprising a computer program product comprising a program code stored thereon, the program code being executable by a processor of an electronic device, the program code comprising instructions that when executed by the processor configure the electronic device to determine a piece of data representing a cerebral marker, said piece of data being obtained from at least one brain network involved in performance of a given task, the determining comprising:
obtaining data on encephalographic activities;
processing the obtained data on encephalographic activities, delivering at least one functional connectivity matrix representing connectivity between cortical sources derived from said data on encephalographic activities, each coefficient of said matrix representing connectivity between two cortical sources;
statistical analysis of said at least one functional connectivity matrix delivering a probabilistic matrix of presence of at least one brain network;
characterizing said at least one brain network on the basis of said at least one functional connectivity matrix and of said statistical analysis, delivering at least one brain network matrix; and
obtaining a cerebral marker as a function of said at least one brain network matrix.

10. The method according to claim 1, further comprising:
measuring encephalographic signals from the at least one brain network involved in the performance of a given task using at least one electrode to obtain the data on encephalographic activities; and
receiving the data on encephalographic activities from the at least one electrode.
